(19) United States
(12) Patent Application Publication
(10) Pub. No.: US 2023/0301835 A1
(43) Pub. Date: Sep. 28, 2023

(54) LOW-GROWTH TISSUE INTERFACE

(71) Applicant: 3M Innovative Properties Company, St. Paul, MN (US)

(72) Inventors: Christopher Brian LOCKE, Bournemouth (GB); Timothy Mark ROBINSON, Shillingstone (GB)

(21) Appl. No.: 18/018,769

(22) PCT Filed: Jul. 20, 2021
(86) PCT No.: PCT/IB2021/056540

§ 371 Date: Jan. 30, 2023

Related U.S. Application Data
(60) Provisional application No. 63/058,969, filed on Jul. 30, 2020.

(51) Int. Cl. A61F 13/00; A61M 1/00

(52) U.S. Cl. CPC A61F 13/00068; A61F 13/00034; A61F 13/00987; A61M 1/915; A61F 13/00021

(57) ABSTRACT

A tissue interface for treating a tissue site and systems and methods of manufacturing the same are described. The tissue interface can include a plurality of shapes and a plurality of ribs. Each rib of the plurality of ribs can have a first end coupled to a respective shape of the plurality of shapes and a second end coupled to at least one other rib of the plurality of ribs. The tissue interface can also include a sheet of felted open-cell foam and a plurality of holes formed in the sheet. Each hole can extend through the sheet. Each hole can have a first end and a second end joined by a gauge section. The first end and the second end can form shoulders that are wider than the gauge section.

### CROSS-REFERENCE TO RELATED APPLICATIONS

This application claims the benefit of priority to U.S. Provisional Application No. 63/058,969, filed on Jul. 30, 2020, which is incorporated herein by reference in its entirety.

### TECHNICAL FIELD

The invention set forth in the appended claims relates generally to tissue treatment systems and more particularly, but without limitation, to tissue interfaces for use in a negative-pressure therapy environment.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for disposition of a tissue interface in a negative-pressure therapy environment are set forth in the appended claims. Illustrative embodiments are also provided to enable a person skilled in the art to make and use the claimed subject matter.

For example, in some embodiments, a tissue interface for treating a tissue site is described. The tissue interface can include a plurality of shapes and a plurality of ribs. Each rib of the plurality of ribs can have a first end coupled to a respective shape of the plurality of shapes and a second end coupled to at least one other rib of the plurality of ribs.

In some embodiments, each shape of the plurality of shapes can be spherical, conical, polyhedral, or cylindrical. In some embodiments, the plurality of shapes are non-uniform. In some embodiments, each shape of the plurality of shapes are perforated. In some embodiments, each shape of the plurality of shapes can comprise a felted open-cell foam or a felted, heat-compressed open-cell foam. A density of the felted foam can be between 5 times and 7 times the density of the un-felted foam. Each shape of the plurality of shapes has a firmness factor between about 5 and about 7. In other embodiments, each shape of the plurality of shapes can have a density of rubber. In other embodiments, the plurality of shapes are formed from a film. In still other embodiments, the plurality of shapes can be formed from a thermoplastic polymer. In some embodiments, the plurality of shapes can be formed from a polymer impregnated textile. In some embodiments, each shape of the plurality of shapes can have an average effective diameter between about 5 mm and about 20 mm.

In some embodiments, the tissue interface can be configured to collapse laterally in response to an application of negative pressure to the tissue interface. In some embodiments, the tissue interface can have a surface area, and the surface area decreases by about 30% in response to an application of negative pressure to the tissue interface.

In some embodiments, the first end of each rib of the plurality of ribs can be tangentially coupled to a surface of the respective shape of the plurality of shapes. Each rib of the plurality of ribs may have a width between about 1 mm and about 4 mm, a thickness up to about 3 mm, and a length between about 1 mm and about 10 mm. In some embodiments, each rib of the plurality of ribs comprises a felted foam. In other embodiments, the plurality of ribs can be formed from a polymeric film. In still other embodiments, the plurality of ribs can be formed from a thermoplastic polymer. In some embodiments, the plurality of ribs can be formed from a polymer impregnated textile. In some embodiments, a plurality of holes can be formed between the plurality of ribs, each hole of the plurality of holes bounded by at least one respective rib of the plurality of ribs.

More generally, a tissue interface for treating a tissue site is described. The tissue interface can include a sheet of felted open-cell foam and a plurality of holes formed in the sheet. Each hole can extend through the sheet. Each hole can have a first end and a second end joined by a gauge section. The first end and the second end can form shoulders that are wider than the gauge section.

Alternatively, other example embodiments may describe a system for treating a tissue site with negative pressure. The system can include a manifold configured to be disposed adjacent to the tissue site. The manifold can have a plurality of nodules and a plurality of webs. Each web of the plurality of webs can have a first end coupled to a respective nodule of the plurality of nodules and a second end coupled to at least one other web of the plurality of webs. The system can further include a sealing member configured to be disposed over the manifold and to seal to tissue surrounding the tissue site. The system can also include a negative-pressure source configured to be fluidly coupled to the manifold and operable to draw fluid through the manifold.

A method of manufacturing a tissue interface is also described. In some example embodiments, a block of open-cell reticulated foam is provided. A pattern can be felted into the block, and portions of the block can be removed. In some embodiments felting the pattern into the block can include forming a plurality of shapes into the block. The block can be compressed, and the block can be heated to permanently increase a density of the block.

In some embodiments, the block can be compressed until a density of the block is between 5 times and 7 times an original density of the block. In some embodiments, the block can be compressed until each shape of the plurality of shapes has a firmness factor between about 5 and about 7. In some embodiments, the block can be compressed until each shape of the plurality of shapes has a density of rubber.

In some embodiments, removing portions of the block may include cutting the block to form a plurality of ribs. Each rib of the plurality of ribs can have a first end coupled to a respective shape of the plurality of shapes and a second end coupled to at least one other rib of the plurality of ribs.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment and instillation treatment in accordance with this specification;

FIG. 2 is a perspective view illustrating additional details of a dressing that may be associated with some embodiments of the therapy system of FIG. 1;

FIG. 3 is a plan view illustrating additional details of a tissue interface that may be associated with some embodiments of the dressing of FIG. 2;

FIG. 4 is a detail view of a portion of the tissue interface of FIG. 3 illustrating additional details that may be associated with some embodiments;

FIG. 5 is a side view illustrating additional details of the tissue interface of FIG. 3 that may be associated with some embodiments;

FIG. 6 is a plan view of the tissue interface of FIG. 2 that may be associated with some embodiments of the tissue interface during the application of negative pressure;

FIG. 7 is a plan view of the dressing of FIG. 2 disposed at a tissue site that may be associated with some embodiments of the therapy system of FIG. 1;

FIG. 8 is sectional view of the dressing of FIG. 2 disposed at another tissue site that may be associated with some embodiments of the therapy system of FIG. 1;

FIG. 9 is a perspective view with a portion shown in section of the dressing of FIG. 8 illustrating additional details that may be associated with some embodiments;

FIG. 10 is a plan view of a portion of another tissue interface illustrating additional details that may be associated with some embodiments of the therapy system of FIG. 1;

FIG. 11 is a side view of the tissue interface of FIG. 10 illustrating additional details that may be associated with some embodiments;

FIG. 12 is a perspective view of the tissue interface of FIG. 10 illustrating additional details that may be associated with some embodiments;

FIG. 13 is a perspective view illustrating additional details of another dressing that may be associated with some embodiments of the therapy system of FIG. 1;

FIG. 14 is a plan view illustrating additional details of a tissue interface that may be associated with some embodiments of the dressing of FIG. 13;

FIG. 15 is a sectional view of the tissue interface of FIG. 13 taken along line 15-15 illustrating additional details that may be associated with some embodiments; and

FIG. 16 is a plan view illustrating additional details of the tissue interface of FIG. 14 that may be associated with some embodiments during application of negative pressure.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

The term “tissue site” in this context broadly refers to a wound, defect, or other treatment target located on or within tissue, including but not limited to, a surface wound, bone tissue, adipose tissue, muscle tissue, neural tissue, dermal tissue, vascular tissue, connective tissue, cartilage, tendons, or ligaments. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted. A surface wound, as used herein, is a wound on the surface of a body that is exposed to the outer surface of the body, such as injury or damage to the epidermis, dermis, and/or subcutaneous layers. Surface wounds may include ulcers or closed incisions, for example. A surface wound, as used herein, does not include wounds within an intra-abdominal cavity. A wound may include chronic, acute, traumatic, subacute, and dehisced wounds, partial-thickness burns, ulcers (such as diabetic, pressure, or venous insufficiency ulcers), flaps, and grafts, for example.

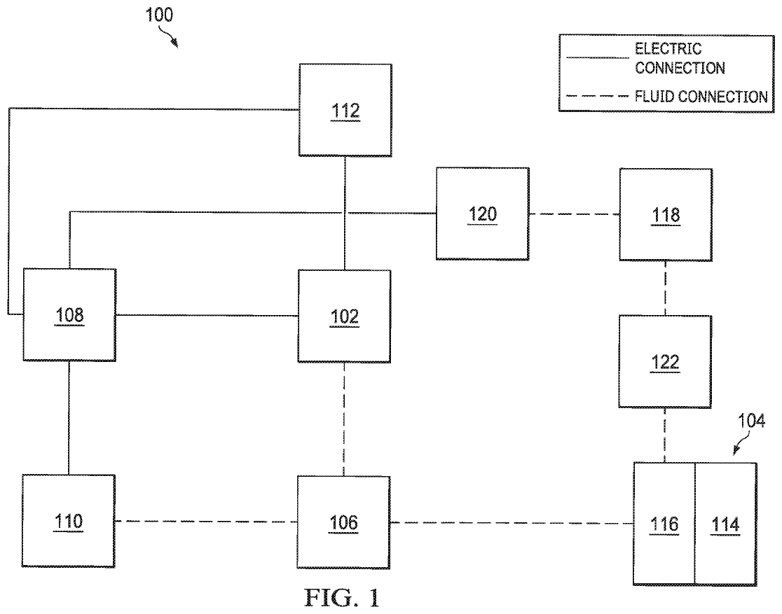

FIG. 1 is a simplified functional block diagram of an example embodiment of a therapy system 100 that can provide negative-pressure therapy with instillation of topical treatment solutions to a tissue site in accordance with this specification. The therapy system 100 may include a source or supply of negative pressure, such as a negative-pressure source 102, a dressing 104, a fluid container, such as a container 106, and a regulator or controller, such as a controller 108, for example. Additionally, the therapy system 100 may include sensors to measure operating parameters and provide feedback signals to the controller 108 indicative of the operating parameters. As illustrated in FIG. 1, for example, the therapy system 100 may include a pressure sensor 110, an electric sensor 112, or both, coupled to the controller 108. As illustrated in the example of FIG. 1, the dressing 104 may comprise or consist essentially of a tissue interface 114, a cover 116, or both in some embodiments.

The therapy system 100 may also include a source of instillation solution. For example, a solution source 118 may be fluidly coupled to the dressing 104, as illustrated in the example embodiment of FIG. 1. The solution source 118 may be fluidly coupled to a positive-pressure source such as the positive-pressure source 120, a negative-pressure source such as the negative-pressure source 102, or both in some embodiments. A regulator, such as an instillation regulator 122, may also be fluidly coupled to the solution source 118 and the dressing 104 to ensure proper dosage of instillation solution (e.g. saline or sterile water) to a tissue site. For example, the instillation regulator 122 may comprise a piston that can be pneumatically actuated by the negative-pressure source 102 to draw instillation solution from the solution source during a negative-pressure interval and to instill the solution to a dressing during a venting interval. Additionally or alternatively, the controller 108 may be coupled to the negative-pressure source 102, the positive-pressure source 120, or both, to control dosage of instillation solution to a tissue site. In some embodiments, the instillation regulator 122 may also be fluidly coupled to the negative-pressure source 102 through the dressing 104, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the solution source 118, the controller 108, and other components into a therapy unit.

In general, components of the therapy system 100 may be coupled directly or indirectly. For example, the negative-pressure source 102 may be directly coupled to the container 106, and may be indirectly coupled to the dressing 104 through the container 106. Coupling may include fluid, mechanical, thermal, electrical, or chemical coupling (such as a chemical bond), or some combination of coupling in some contexts. For example, the negative-pressure source 102 may be electrically coupled to the controller 108, and may be fluidly coupled to one or more distribution components to provide a fluid path to a tissue site. In some embodiments, components may also be coupled by virtue of physical proximity, being integral to a single structure, or being formed from the same piece of material. For example, the tissue interface 114 and the cover 116 may be discrete layers disposed adjacent to each other, and may be joined together in some embodiments.

A distribution component is preferably detachable, and may be disposable, reusable, or recyclable. The dressing 104 and the container 106 are illustrative of distribution components. A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 104.

A negative-pressure supply, such as the negative-pressure source 102, may be a reservoir of air at a negative pressure, or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 106 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 108, may be a microprocessor or computer programmed to operate one or more components of the therapy system 100, such as the negative-pressure source 102. In some embodiments, for example, the controller 108 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 114, for example. The controller 108 is also preferably configured to receive one or more input signals, such as a feedback signal, and programmed to modify one or more operating parameters based on the input signals.

Sensors, such as the pressure sensor 110 or the electric sensor 112, are generally known in the art as any apparatus operable to detect or measure a physical phenomenon or property, and generally provide a signal indicative of the phenomenon or property that is detected or measured. For example, the pressure sensor 110 and the electric sensor 112 may be configured to measure one or more operating parameters of the therapy system 100. In some embodiments, the pressure sensor 110 may be a transducer configured to measure pressure in a pneumatic pathway and convert the measurement to a signal indicative of the pressure measured. In some embodiments, the pressure sensor 110 may be a piezoresistive strain gauge. The electric sensor 112 may optionally measure operating parameters of the negative-pressure source 102, such as the voltage or current, in some embodiments. Preferably, the signals from the pressure sensor 110 and the electric sensor 112 are suitable as an input signal to the controller 108, but some signal conditioning may be appropriate. For example, the signal may need to be filtered or amplified before it can be processed by the controller 108. Typically, the signal is an electrical signal, but may be represented in other forms, such as an optical signal.

The tissue interface 114 can be generally adapted to partially or fully contact a tissue site. The tissue interface 114 may take many forms, and may have many sizes, shapes, or thicknesses depending on a variety of factors, such as the type of treatment being implemented or the nature and size of a tissue site. For example, the size and shape of the tissue interface 114 may be adapted to the contours of deep and irregular shaped tissue sites.

In some embodiments, the cover 116 may provide a bacterial barrier and protection from physical trauma. The cover 116 may also be constructed from a material that can reduce evaporative losses and provide a fluid seal between two components or two environments, such as between a therapeutic environment and a local external environment. The cover 116 may be, for example, an elastomeric film or membrane that can provide a seal adequate to maintain a negative pressure at a tissue site for a given negative-pressure source. The cover 116 may have a high moisture-vapor transmission rate (MVTR) in some applications. For example, the MVTR may be at least about 300 g/m² per twenty-four hours in some embodiments. In some example embodiments, the cover 116 may be a polymer drape, such as a polyurethane film, that is permeable to water vapor but impermeable to liquid. Such drapes typically have a thickness in the range of about 25 microns to about 50 microns. For permeable materials, the permeability generally should be low enough that a desired negative pressure may be maintained.

The cover 116 may comprise, for example, one or more of the following materials: hydrophilic polyurethane; cellulosics; hydrophilic polyamides; polyvinyl alcohol; polyvinyl pyrrolidone; hydrophilic acrylics; hydrophilic silicone elastomers; an INSPIRE 2301 material from Coveris Advanced Coatings of Wrexham, United Kingdom having, for example, an MVTR (inverted cup technique) of about 14400 g/m²/24 hours and a thickness of about 30 microns; a thin, uncoated polymer drape; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; polyurethane (PU); EVA film; co-polyester; silicones; a silicone drape; a 3M Tegaderm® drape; a polyurethane (PU) drape such as one available from Avery Dennison Corporation of Glendale, California; polyether block polyamide copolymer (PEBAX), for example, from Arkema, France; INSPIRE 2327; or other appropriate material.

An attachment device may be used to attach the cover 116 to an attachment surface, such as undamaged epidermis, a gasket, or another cover. The attachment device may take many forms. For example, an attachment device may be a medically-acceptable, pressure-sensitive adhesive configured to bond the cover 116 to epidermis around a tissue site. In some embodiments, for example, some or all of the cover 116 may be coated with an adhesive, such as an acrylic adhesive, which may have a coating weight between about 25 grams per square meter (g·s·m.) and about 65 g·s·m. Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 118 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies a position in a fluid path relatively closer to a source of negative pressure or further away from a source of positive pressure. Conversely, the term “upstream” implies a position relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

During treatment of a tissue site, some tissue sites may not heal according to the normal medical protocol and may develop areas of necrotic tissue. Necrotic tissue may be dead tissue resulting from infection, toxins, or trauma that caused the tissue to die faster than the tissue can be removed by the normal body processes that regulate the removal of dead tissue. Sometimes, necrotic tissue may be in the form of slough, which may include a viscous liquid mass of tissue. Generally, slough is produced by bacterial and fungal infections that stimulate an inflammatory response in the tissue. Slough may be a creamy yellow color and may also be referred to as pus. Necrotic tissue may also include eschar. Eschar may be a portion of necrotic tissue that has become dehydrated and hardened. Eschar may be the result of a burn injury, gangrene, ulcers, fungal infections, spider bites, or anthrax. Eschar may be difficult to remove without the use of surgical cutting instruments.

In addition to necrotic tissue, slough, and eschar, the tissue site may include biofilms, lacerated tissue, devitalized tissue, contaminated tissue, damaged tissue, infected tissue, exudate, highly viscous exudate, fibrinous slough and/or other material that can generally be referred to as debris. The debris may inhibit the efficacy of tissue treatment and slow the healing of the tissue site. If the debris is in the tissue site, the tissue site may be treated with different processes to disrupt the debris. Examples of disruption can include softening of the debris, separation of the debris from desired tissue, such as the subcutaneous tissue, preparation of the debris for removal from the tissue site, and removal of the debris from the tissue site.

The debris can require debridement performed in an operating room. In some cases, tissue sites requiring debridement may not be life-threatening, and debridement may be considered low-priority. Low-priority cases can experience delays prior to treatment as other, more life-threatening, cases may be given priority for an operating room. As a result, low priority cases may need temporization. Temporization can include stasis of a tissue site that limits deterioration of the tissue site prior to other treatments, such as debridement, negative-pressure therapy or instillation.

When debriding, clinicians may find it difficult to define separation between healthy, vital tissue and necrotic tissue. As a result, normal debridement techniques may remove too much healthy tissue or not enough necrotic tissue. If non-viable tissue demarcation does not extend deeper than the deep dermal layer, or if the tissue site is covered by the debris, such as slough or fibrin, gentle methods to remove the debris should be considered to avoid excess damage to the tissue site.

In some debridement processes, a mechanical process is used to remove the debris. Mechanical processes may include using scalpels or other cutting tools having a sharp edge to cut away the debris from the tissue site. Other mechanical processes may use devices that can provide a stream of particles to impact the debris to remove the debris in an abrasion process, or jets of high pressure fluid to impact the debris to remove the debris using water jet cutting or lavage. Typically, mechanical processes of debriding a tissue site may be painful and may require the application of local anesthetics. Mechanical processes also risk over removal of healthy tissue that can cause further damage to the tissue site and delay the healing process.

Debridement may also be performed with an autolytic process. For example, an autolytic process may involve using enzymes and moisture produced by a tissue site to soften and liquefy the necrotic tissue and debris. Typically, a dressing may be placed over a tissue site having debris so that fluid produced by the tissue site may remain in place, hydrating the debris. Autolytic processes can be pain-free, but autolytic processes are a slow and can take many days. Because autolytic processes are slow, autolytic processes may also involve many dressing changes. Some autolytic processes may be paired with negative-pressure therapy so that, as debris hydrates, negative pressure supplied to a tissue site may draw off the debris. In some cases, a manifold positioned at a tissue site to distribute negative pressure across the tissue site may become blocked or clogged with debris broken down by an autolytic process. If a manifold becomes clogged, negative pressure may not be able to remove debris, which can slow or stop the autolytic process.

Debridement may also be performed by adding enzymes or other agents to the tissue site that digest tissue. Often, strict control of the placement of the enzymes and the length of time the enzymes are in contact with a tissue site must be maintained. If enzymes are left on a tissue site for longer than needed, the enzymes may remove too much healthy tissue, contaminate the tissue site, or be carried to other areas of a patient. Once carried to other areas of a patient, the enzymes may break down undamaged tissue and cause other complications.

Furthermore, some dressings may have limited capability to receive and distribute fluid. For example, the material of a tissue interface of a dressing may have a density that limits or prevents fluid flow through the tissue interface. That same tissue interface may have large perforations through the tissue interface. The large perforations can permit fluid to flow across the tissue interface with little resistance or no resistance. The variation between dense regions and perforations of the tissue interface may create different fluid flow rates across the tissue interface. The difference in fluid flow rates across the tissue interface may create high pressure gradients across the tissue interface. A high-pressure gradient may concentrate fluid flow at the tissue site to the sections of high fluid flow through the perforations. Concentration of fluid flow may limit lateral fluid flow across the tissue site between the tissue site and the tissue interface. As a result, fluid flow across the tissue site may be restricted at the surface of the tissue site in contact with the dressing.

Some dressings may have a tissue interface formed from a stiff material. These tissue interfaces may be difficult to fold or bend. As a result, the ability of the tissue interface to conform to complex curves may be limited. For example, a stiff tissue interface may have difficulty being bent around an arm or, more particularly, an elbow, limiting the ability of the dressing to be used at these types of locations. Still further, the stiffness of the material can inhibit the ability of the material to be re-sized for smaller tissue sites. For example, a tissue interface may have a first size and need to be cut or torn into a second smaller size to fit into a tissue site. A tissue interface having a density that limits fluid flow through the material of the tissue interface may resist cutting or tearing. As a result, the user may have difficulty placing the tissue interface at the tissue site.

These limitations and others may be addressed by the therapy system 100, which can provide negative-pressure therapy, instillation therapy, and disruption of debris. In some embodiments, the therapy system 100 can provide mechanical movement at a surface of the tissue site in combination with cyclic delivery and dwell of topical solutions to help solubilize debris. For example, a negative-pressure source may be fluidly coupled to a tissue site to provide negative pressure to the tissue site for negative-pressure therapy. In some embodiments, a fluid source may be fluidly coupled to a tissue site to provide therapeutic fluid to the tissue site for instillation therapy. In some embodiments, the therapy system 100 may include a contact layer positioned adjacent to a tissue site that may be used with negative-pressure therapy, instillation therapy, or both to disrupt areas of a tissue site having debris. Following the disruption of the debris, negative-pressure therapy, instillation therapy, and other processes may be used to remove the debris from a tissue site. In some embodiments, the therapy system 100 may be used in conjunction with other tissue removal and debridement techniques. For example, the therapy system 100 may be used prior to enzymatic debridement to soften the debris. In another example, mechanical debridement may be used to remove a portion of the debris at the tissue site, and the therapy system 100 may then be used to remove the remaining debris while reducing the risk of trauma to the tissue site. The therapy system 100 may also provide a dressing that may improve fluid flow across a surface of a tissue site, the total ability of the tissue interface to move fluid from the tissue site, conformability to a tissue site, and customization for use at tissue sites of varying sizes and shapes, thereby increasing the effectiveness of the therapy system 100. Still other embodiments of the therapy system 100 may provide a dressing that can at least partially collapse under negative pressure, generating apposition forces that may draw edges of the tissue site together.

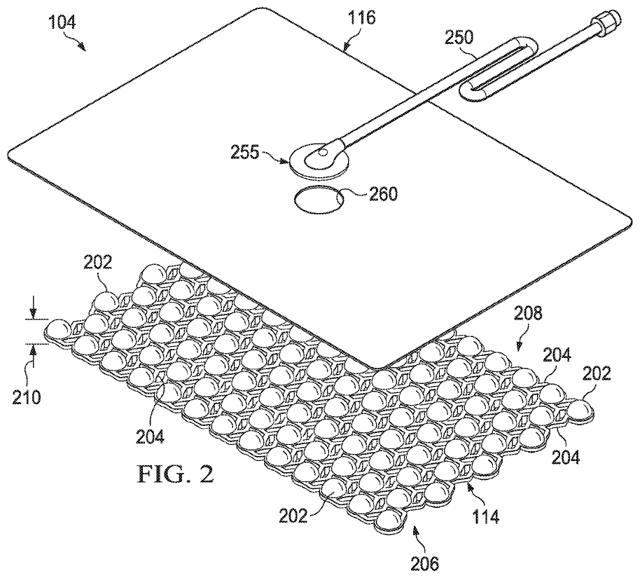

FIG. 2 is an assembly view of an example of the dressing 104 of FIG. 1, illustrating additional details that may be associated with some embodiments. For example, the tissue interface 114 may be a manifold, a contact layer, or a debridement tool. The tissue interface 114 may have a plurality of shapes or nodules 202 coupled to each other by a web. In some embodiments, the web can comprise a plurality of ribs 204. The plurality of ribs 204 may be coupled to the plurality of nodules 202 and each other to form the tissue interface 114. In some embodiments, the tissue interface 114 may have a first side 206, a second side 208, and a thickness 210 from the first side 206 to the second side 208.

As illustrated in the example of FIG. 2, in some embodiments, the dressing 104 may include a fluid conductor 250 and a dressing interface 255. As shown in the example of FIG. 2, the fluid conductor 250 may be a flexible tube, which can be fluidly coupled on one end to the dressing interface 255. The dressing interface 255 may be an elbow connector, as shown in the example of FIG. 2, which can be placed over an aperture 260 in the cover 116 to provide a fluid path between the fluid conductor 250 and the tissue interface 114. In some embodiments, the tissue interface 114 may be provided as a portion of an assembly forming the dressing 104. In other embodiments, the tissue interface 114 may be provided separately from the cover 116, the fluid conductor 250, and the dressing interface 255 for assembly of the dressing 104 at the point of use.

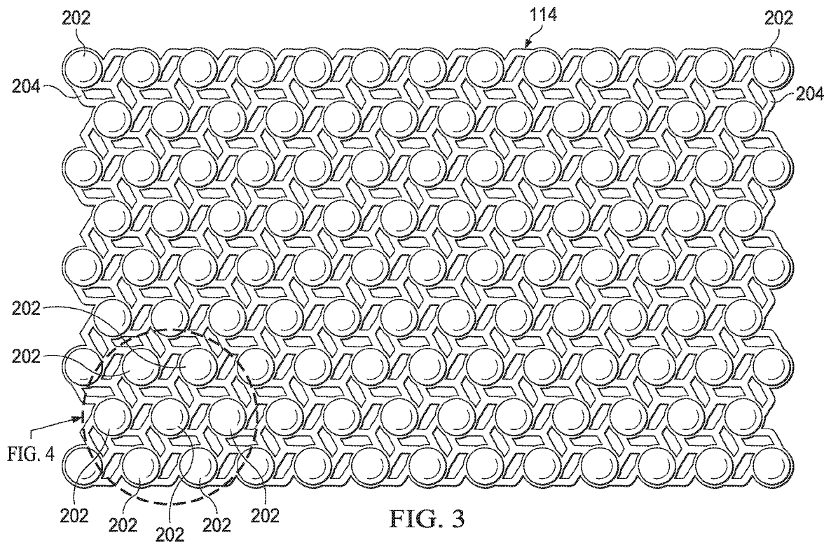

FIG. 3 is a plan view of the tissue interface 114 illustrating additional details that may be associated with some embodiments. The tissue interface 114 may be formed from a foam, for example, an open-cell, reticulated foam that is compressed and/or felted into shaped sections. For example, cellular foam, open-cell foam, reticulated foam, or porous tissue collections, may be used to form the tissue interface 114. In some embodiments, the tissue interface 114 may be a foam having pore sizes in a range of about 60 microns to about 2000 microns. In other embodiments, the tissue interface 114 may be a foam having pore sizes in a range of about 400 microns to about 600 microns. The tensile strength of the tissue interface 114 may vary according to needs of a prescribed therapy. For example, the tensile strength of a foam may be increased for instillation of topical treatment solutions. The 25% compression load deflection of the tissue interface 114 may be at least 0.35 pounds per square inch, and the 65% compression load deflection may be at least 0.43 pounds per square inch. In some embodiments, the tensile strength of the tissue interface 114 may be at least 10 pounds per square inch. The tissue interface 114 may have a tear strength of at least 2.5 pounds per inch. In one non-limiting example, the tissue interface 114 may be an open-cell, reticulated polyurethane foam such as V.A.C.® GRANUFOAM™ Dressing available from Kinetic Concepts, Inc. of San Antonio, Texas; in other embodiments the tissue interface 114 may be an open-cell, reticulated polyurethane foam such as a V.A.C. VERAFLO™ dressing, also available from Kinetic Concepts, Inc., of San Antonio, Texas. In other embodiments, the tissue interface 114 may be formed of an un-reticulated open-cell foam.

In some embodiments, the tissue interface 114 may be formed from a foam that is mechanically or chemically compressed, often as part of a thermoforming process, to increase the density of the foam at ambient pressure. A foam that is mechanically or chemically compressed may be referred to as a compressed foam or a felted foam. A compressed foam may be characterized by a firmness factor (FF) that is defined as a ratio of the density of a foam in a compressed state to the density of the same foam in an uncompressed state. For example, a firmness factor (FF) of 5 may refer to a compressed foam having a density at ambient pressure that is five times greater than a density of the same foam in an uncompressed state at ambient pressure. Generally a compressed or felted foam may have a firmness factor greater than 1.

Mechanically or chemically compressing a foam may reduce a thickness of the foam at ambient pressure when compared to the same foam that has not been compressed. Reducing a thickness of a foam by mechanical or chemical compression may increase a density of the foam, which may increase the firmness factor (FF) of the foam. Increasing the firmness factor (FF) of a foam may increase a stiffness of the foam in a direction that is parallel to a thickness of the foam. For example, increasing a firmness factor (FF) of the tissue interface 114 may increase a stiffness of the tissue interface 114 in a direction that is parallel to the thickness 210 of the tissue interface 114. In some embodiments, a compressed foam may be a compressed V.A.C.® GRANUFOAM™ Dressing. V.A.C.® GRANUFOAM™ Dressing may have a density of about 0.03 grams per centimeter³ (g/cm³) in its uncompressed state. If the V.A.C.® GRANUFOAM™ Dressing is compressed to have a firmness factor (FF) of 5, the V.A.C.® GRANUFOAM™ Dressing may be compressed until the density of the V.A.C.® GRANUFOAM™ Dressing is about 0.15 g/cm³. V.A.C. VERAFLO™ dressing may also be compressed to form a compressed foam having a firmness factor (FF) up to 5. For example, V.A.C. VERAFLO™ Dressing, may have a density between about 1.7 pounds per foot³ (lb/ft³) or 0.027 grams per centimeter³ (g/cm³) and about 2.1 lb/ft³ or 0.034 g/cm³. If the V.A.C. VERAFLO™ Dressing is compressed to have a firmness factor (FF) of 5, the V.A.C. VERAFLO™ Dressing may be compressed until the density of the V.A.C. VERAFLO™ Dressing is between about 0.135 g/cm³ and about 0.17 g/cm³.

Generally, if a compressed foam is subjected to negative pressure, the compressed foam exhibits less deformation than a similar uncompressed foam. If the tissue interface 114 is formed of a compressed foam, the thickness 210 of the tissue interface 114 may deform less than if the tissue interface 114 is formed of a comparable uncompressed foam. The decrease in deformation may be caused by the increased stiffness as reflected by the firmness factor (FF). If subjected to the stress of negative pressure, the tissue interface 114 that is formed of compressed foam may flatten less than the tissue interface 114 that is formed from uncompressed foam. Consequently, if negative pressure is applied to the tissue interface 114, the stiffness of the tissue interface 114 in the direction parallel to the thickness 210 of the tissue interface 114 allows the tissue interface 114 to be more compliant or compressible in other directions, e.g., a direction perpendicular to the thickness 210. The foam material used to form a compressed foam may be either hydrophobic or hydrophilic. The foam material used to form a compressed foam may also be either reticulated or un-reticulated. The pore size of a foam material may vary according to needs of the tissue interface 114 and the amount of compression of the foam. For example, in some embodiments, an uncompressed foam may have pore sizes in a range of about 400 microns to about 600 microns. If the same foam is compressed, the pore sizes may be smaller than when the foam is in its uncompressed state. In some embodiments, the tissue interface 114 can be manufactured by providing a foam block. The foam block may be felted or otherwise permanently deformed to increase a density of the foam block to the desired density.

The coupling of the ribs 204 to each other and the nodules 202 to comprise the tissue interface 114 forms a structure configured to manifold fluids. In some embodiments, the material of the ribs 204 may be felted so that the void space percentage of the material approaches zero. The void space percentage of a material can refer to the percentage of the volume of the material, for example a foam material, that is formed by a gas, such as ambient air. A material having a void space percentage of zero has no gas content in the volume of the material. A material having a void space percentage of one hundred has no solid content in the volume of the material. The spacing and the total number of the ribs 204 may allow the tissue interface 114 to manifold fluid, distort in shape, and collapse in a lateral direction parallel to a primary plane of the tissue interface 114. In some embodiments, each nodule 202 of the plurality of nodules 202 may be coupled to at least one other nodule 202 by at least one rib 204. For example, each nodule 202 may generally have six ribs 204 extending from the nodule 202. The six ribs 204 may form a portion of a radial web or distortable web. Other nodules 202, for example, those disposed at an edge of the tissue interface 114 may have fewer ribs 204 coupled to the nodule 202. In some embodiments, the ribs 204 may be tangential to a nodule 202 and intersect at least one rib 204 of an adjacent nodule 202. In other embodiments, the ribs 204 may be normal to an effective diameter of a respective nodule 202.

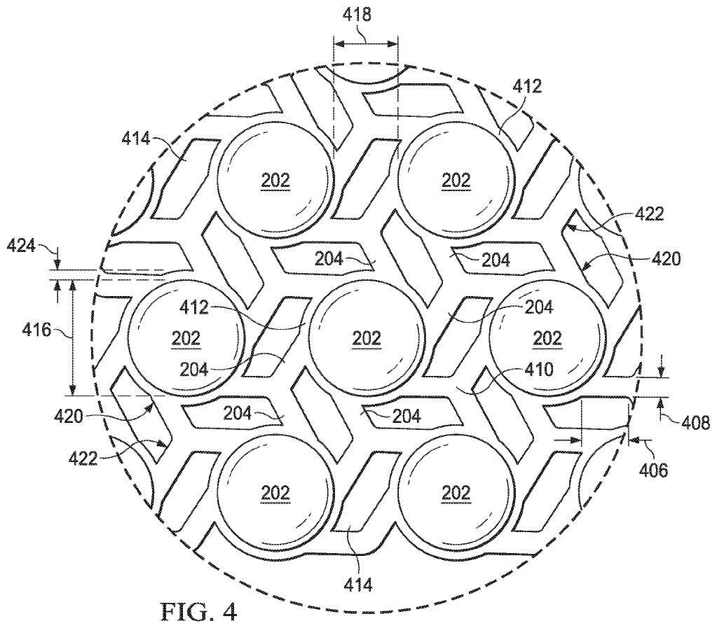

FIG. 4 is a detail view of a portion of the tissue interface 114 of FIG. 3, illustrating additional details that may be associated with some embodiments. The relationship between adjacent nodules 202 of the plurality of nodules 202 can be described with reference to the portion of the tissue interface 114 illustrated in FIG. 4. The portion of the tissue interface 114 may contain a grouping of seven nodules 202. In some embodiments, the grouping of nodules 202 may include a nodule 202 surrounded by six other nodules 202. A grouping of nodules 202 centered on a nodule 202 that may be located at an edge of the tissue interface 114 may have fewer nodules 202. In the illustrated embodiment, a nodule 202 located at an edge of the tissue interface 114 may be surrounded four nodules 202 or if the nodule 202 is located at the intersection of two edges, two nodules 202. In some embodiments, the nodules 202 can be equidistantly spaced from adjacent nodules 202. For example, adjacent nodules 202 may have a distance 418 between edges of the nodules 202 at a surface of the ribs 204. The distance 418 may be the shortest distance between the surfaces of adjacent nodules 202. In some embodiments, the distance 418 can be between about 3 mm and about 8 mm and, preferably, about 5 mm. Preferably, the nodules 202 and the ribs 204 are disposed in a same plane.

In some embodiments, each nodule 202 may be spherical. In other embodiments, the nodules 202 can be polyhedra, cylinders, cones, amorphous shapes, or mixtures of multiple shapes. For example, a tissue interface 114 may include nodules 202 having spherical, cylindrical, conical, and polyhedral shapes. Each of the nodules 202 can have an effective diameter 416 between about 5 mm and about 20 mm. In some embodiments, a maximum thickness of the tissue interface 114, the thickness 210, may be equal to the diameter 416 of the nodules 202.

In some embodiments, each nodule 204 can be surrounded by a nodule ring 412. The nodule ring 412 may be a ring of material surrounding the nodule 204. In some embodiments, the ribs 204 may couple to the nodule ring 412. The nodule ring 412 may have a radial width 424 from an exterior surface of the nodule 202 to an edge of the nodule ring 412 of about 0.5 mm. Each nodule ring 412 may have a thickness equal to a thickness of the plurality of ribs 204. Preferably, the nodule ring 412 is disposed at about an equator of the nodule 202 associated with the nodule ring 412.

Each nodule 202 can include at least six ribs 204 coupled to the nodule 202. The ribs 204 can be equidistantly spaced around the nodule 202. In some embodiments, each rib 204 may be coupled to a nodule 202 so that the rib 204 is tangential to the nodule 204. In other embodiments, the ribs 204 are normal to the nodules 202 or extend radially from the nodule 202. Each rib 204 can have a length 406 and a width 408. Each rib 204 may have a long side 420 intersecting an exterior edge of the respective nodule ring 214 substantially tangential to the nodule ring 214 and the nodule 202. Each rib 204 can also have a short side 422 intersecting the exterior edge of the respective nodule ring 214 and forming an acute angle with a long side 420 of a counter-clockwise adjacent rib 204 of the nodule 202. The length 406 can refer to the long side 420 of the rib 204. In some embodiments, the length 406 may be between about 1 mm to about 10 mm. The width 408 may be between about 1 mm and about 4 mm.

Each rib 204 may intersect at least one other rib 204 extending from an adjacent nodule 204. In some embodiments, a distal end of each rib 204 can meet and be coupled to a distal end of at least two other ribs 204 each extending from a separate nodule 204. In some embodiments, the junction of ribs 204 can form a rib node 410. In some embodiments, the ribs 204 coupled to each other at a rib node 410 can be equidistantly spaced from each other. For example, each rib 204 may form an angle of about 120 degrees with the ribs 204 to which the rib 204 is coupled at a rib node 410. In other embodiments, the ribs 204 coupled to each other at a rib node 410 may not be equidistantly spaced from each other.

The ribs 204 can space the nodules 202 from each other, forming a plurality of openings 414 between the ribs 204 and the nodules 202. In some embodiments, the openings 414 may allow the passage of fluid from the tissue site, including exudates and gases. The openings 414 may further permit the nodules 202 to move relative to each other. In some embodiments, each opening 414 may have an area between about 2 mm² and about 40 mm². In some embodiments, the openings 414 may have a pitch about equal to the diameter 416 of a nodule 202 plus the width 408 of a rib 204. For example, a pitch of the openings 414 may be about 6 mm and about 24 mm. The openings 414 may create a void space within the tissue interface 114. The void space may refer to the portion of the volume of the tissue interface 114 that is non-solid material, for example, the void space can refer to the portion of the tissue interface 114 that is formed by the openings 414 rather than the nodules 202 and the ribs 204. In some embodiments, the void space may be about 30% to about 40% of the total volume of the tissue interface 114 when the tissue interface 114 is uncompressed.

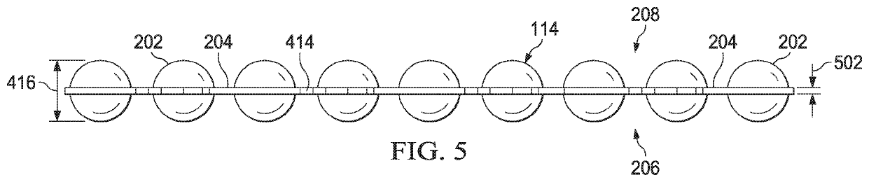

FIG. 5 is a side view of the tissue interface 114, illustrating additional details that may be associated with some embodiments. The ribs 204 may have a thickness 502. In some embodiments, the thickness 502 may be up to about 3 mm. In some embodiments, the ribs 204 may couple adjacent nodules 202 to each other adjacent to equators of the nodules 202 so that each nodule 202 extends equidistantly from opposing surfaces of the ribs 204. For example, if the diameter 416 of the nodule 202 is about 10 mm, about 5 mm of the nodule 202 may project onto the first side 206 and about 5 mm of the nodule 202 may project onto the second side 208.

In some embodiments, the tissue interface 114 can be formed by felting the patterns into sections of a base block of foam that is heated and forms both the nodules 202 and a web between the nodules. Following the felting process, a cutting tool can be used to perforate the pattern of the openings 414 to form the ribs 204. The perforated material can be extracted by a high flow vacuum system. For example, a block of open-cell, reticulated foam can be provided, a pattern can be felted into the block, and portions of the block can be removed. In some embodiments, felting the pattern into the block can include forming a plurality of shapes into the block, compressing the block, and heating the block to permanently increase a density of the block. In some embodiments, a form or mold can be used. The mold can create both the nodules and a continuous web between the nodules. The mold can create different densities within the tissue interface 114. For example, the mold can be a bi-valve mold having hollows in each half corresponding to the nodules 202. The mold can be applied to opposing surfaces of a block of un-felted foam. The mold can compress the foam while heating the foam. Following compression and heating, the nodules 202 may be joined to each other by a continuous web of highly-felted foam material. In some embodiments, the nodules 202 may have a density about 5 to about 7 times the original density of the foam block. The form can felt the block at the web between the nodules 202 to have a maximum density of the material of the block. For example, if the material of the block is V.A.C.® GRANUFOAM™ Dressing, the form can felt the material to a firmness factor of at least 7 giving the block a resulting density of about 0.168 g/cm³. In some embodiments, the block can be compressed the continuous web has a density of a polyurethane elastomer, a rubber, or a film. For example, the continuous web can have a density of about 1.522 g/cm³. For an open-cell reticulated foam, such as V.A.C.® GRANUFOAM™ Dressing, the felting level may be about 63 times the original density of the foam. Following the felting process, the continuous web can be cut, for example, by die-cutting, to form the openings 414. In some embodiments, the plurality of shapes are non-uniform.

In some embodiments, removing portions of the block comprises cutting the block to form a plurality of ribs, each rib of the plurality of ribs having a first end coupled to a respective shape of the plurality of shapes and a second end coupled to at least one other rib of the plurality of ribs. In some embodiments, the first end of each rib of the plurality of ribs is tangentially coupled to a surface of the respective shape of the plurality of shapes. In some embodiments, each rib of the plurality of ribs may have a width between about 1 mm and about 4 mm, a thickness up to about 3 mm, and a length between about 1 mm and about 10 mm.

In some embodiments, the tissue interface 114 can be formed with a closed cell foam, such as Zote foam that is molded or vacuum formed to provide a similar structure. In some embodiments, the nodules 202 formed from closed cell foam can be perforated to permit fluid flow through the nodules 202. In other embodiments, the tissue interface 114 can be formed from a gas inflated film. In still other embodiments, the tissue interface can be formed by casting or molding the tissue interface from thermoplastic polymers. In some embodiments, the tissue interface 114 can be formed from impregnated textiles or other non-wovens that are heat molded into a form as described and illustrated herein. In some embodiments, the openings 414 can be cut into the tissue interface 114 having a shape that permits the openings 414 to remain at least partially open when under negative pressure. For example, the openings 414 may be cut so that intersecting surfaces between a length 406 and a width 408 of the ribs 204 do not meet at an angle forming an edge. In some embodiments, intersecting surfaces between the length 406 and the width 408 of an opening 414 may have a radius of curvature.

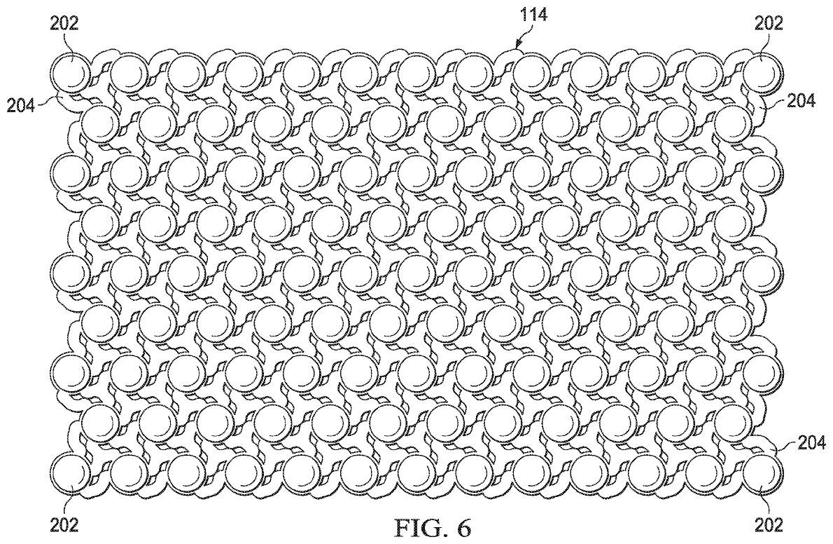

FIG. 6 is a plan view of the tissue interface 114 illustrating additional details that may be associated with some embodiments of the tissue interface 114 under negative pressure. In some embodiments, the nodules 202, the ribs 204, and the openings 414 cooperate to collapse in response to the application of negative pressure. For example, if the tissue interface 114 is disposed at a tissue site and subjected to a negative pressure of about 125 mm Hg, the tissue interface 114 may reduce in area between about 15% and about 30%. Compression of the tissue interface 114 may reduce the void space of the tissue interface 114 formed by the openings 414 to about 10% to about 20% of the total volume of the tissue interface 114. An amount of collapse can be controlled in part by controlling a size of the openings 414. For example, increasing a size of the openings 414 by increasing a width of the openings 414 can decrease the width 408 of the ribs 204 and increasing the reduction in area of the tissue interface 114 under negative pressure. Conversely, decreasing a size of the openings 414 by decreasing a width of the openings 414 can increase the width 408 of the ribs 204 and decreasing the reduction in area of the tissue interface 114 under negative pressure. In some embodiments, the openings 414 may collapse so that a long side 420 of a first rib 204 coupled to a first nodule 202 substantially collapses against a long side 420 of a second rib 204 coupled to a second nodule 202, generating a radial collapse of the tissue interface 114. A radial collapse of the tissue interface 114 may cause both a length and a width of the tissue interface 114 to collapse. The long side 420 of the second rib 204 may be facing the long side 420 of the first rib 204. In some embodiments, the collapse of the tissue interface 114 may generate an apposition force urging edges of the tissue site toward each other.

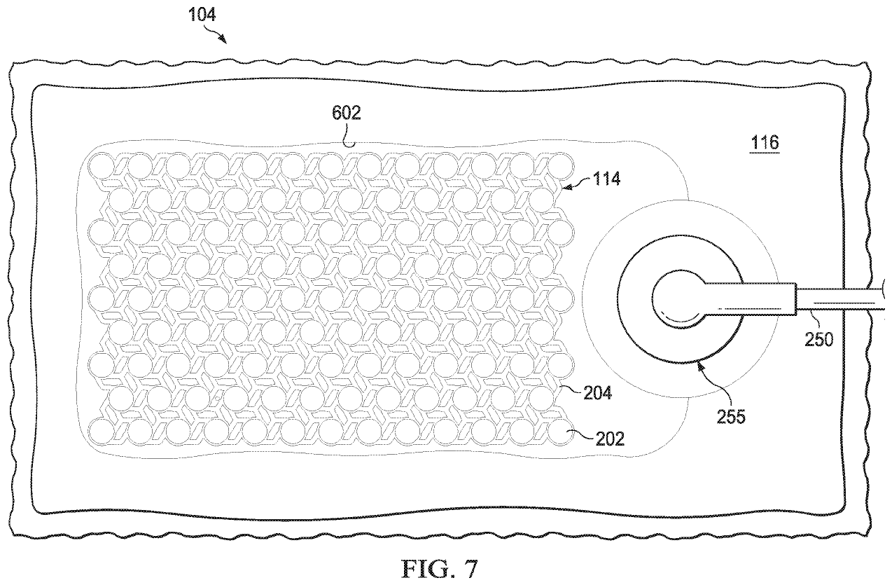

FIG. 7 is a plan view of the dressing 104 disposed at a tissue site 602 illustrating additional details that may be associated with some embodiments. In some embodiments, the tissue interface 114 can be disposed at a tissue site so that a primary plane of the tissue interface 114 is parallel to a plane of the tissue site. In some embodiments, the nodules 202 and the ribs 204 may be arranged to easily facilitate cutting with scissors or a scalpel, permitting straight lines to be formed along the ribs 204. This allows a user to form a tissue interface appropriately sized for the tissue site 602 from a singular larger tissue interface. In some embodiments, the tissue interface 114 can be sized by cutting the tissue interface 114 at the ribs 204. For example, the ribs 204 may be severed from each other by cutting or tearing, allowing the tissue interface 114 to be reduced in size. The tissue interface 114 can then be disposed at a tissue site, for example, the tissue site 602. The cover 116 can be disposed over the tissue interface 114 and the tissue site 602 and sealed to undamaged epidermis around the tissue site 602. The connector 255 can be coupled to the cover 116 and negative-pressure and/or instillation fluid can be supplied to the tissue site 602 and the tissue interface 114 through the fluid conductor 250 and the connector 255. In an exemplary operation, negative pressure can be supplied to the tissue site 602. As fluid is drawn from the tissue site 602 through the tissue interface 114, the openings 414 may at least partially collapse. In response, the tissue interface 114 may contract, generating an apposition force that urges edges of a tissue site 602 toward each other. In some embodiments, a cover layer can be disposed over the tissue interface 114 to aid in filling the tissue site 602. For example, a layer of foam material may be disposed over the tissue interface 114, covering the tissue interface 114. Generally, contact between the tissue interface 114 and undamaged epidermis adjacent to the tissue site 602 may be avoided.

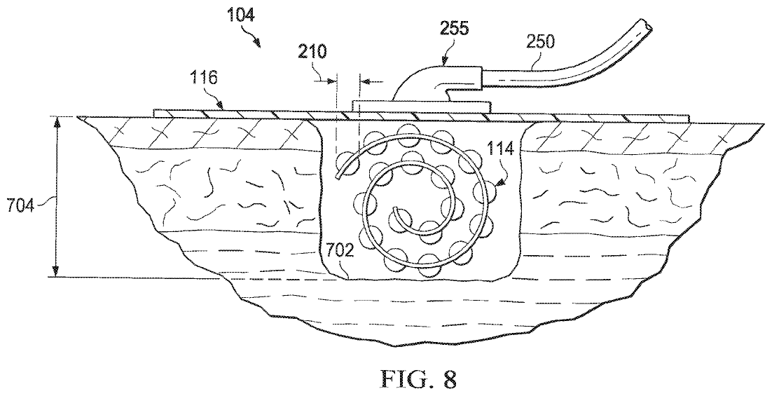

FIG. 8 is a sectional view of the dressing 104 disposed at another tissue site 702. And FIG. 9 is a perspective view with a portion shown in section of the dressing 104 disposed at the tissue site 702. In some embodiments, the tissue interface 114 can be folded or rolled to be disposed at a tissue site or disposed into a deep or tunneled tissue site. In some embodiments, the tissue site 702 may have a depth 704 greater than the thickness 210 of the tissue interface 114. To size the tissue interface 114 to fit the depth 704 of the tissue site 702, the tissue interface 114 may be rolled over an edge of the tissue interface 114 to form a cylinder like body. The tissue interface 114 can then be inserted into the tissue site 702 to fill the depth 704. The tissue interface 114 may be rolled over an edge of the tissue interface 114 due to the flexibility imparted to the tissue interface 114 by the ribs 204. For example, the thickness and pliability of the ribs 204 permit the tissue interface 114 to be contoured. In some embodiments, the ribs 204 may permit adjacent rows of the nodules 204 to be disposed over each other without uncoupling the nodules 204 from each other.

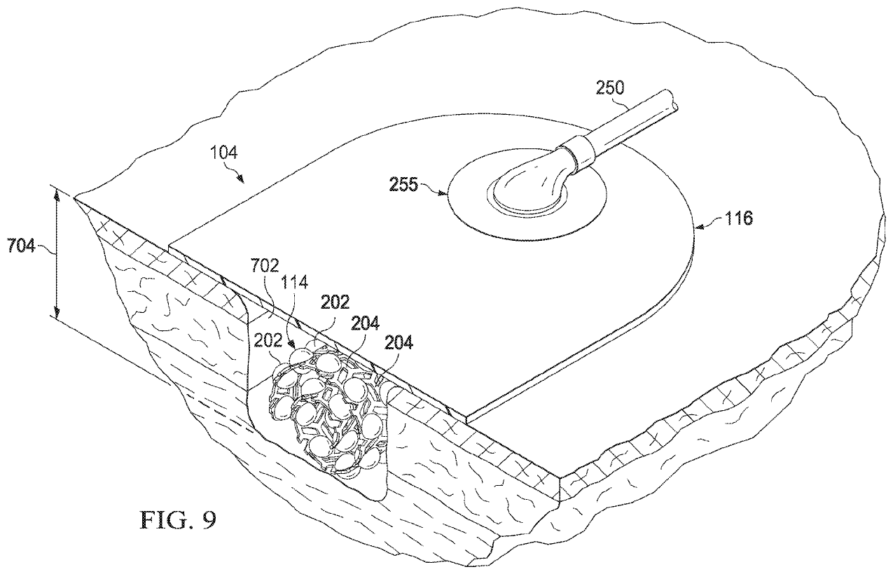

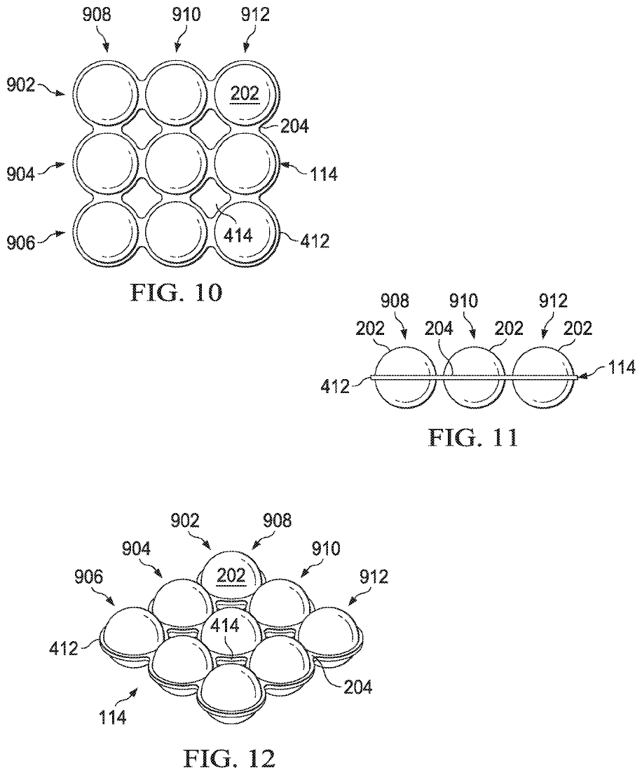

FIG. 10 is a plan view of a portion of another tissue interface illustrating additional details that may be associated with some embodiments of the therapy system of FIG. 1. FIG. 11 is a side view of the tissue interface of FIG. 10 illustrating additional details that may be associated with some embodiments. And FIG. 12 is a perspective view of the tissue interface of FIG. 10 illustrating additional details that may be associated with some embodiments. In some embodiments, the nodules 202 can be arranged in parallel rows. For example, the nodules 202 can be arranged in a first row 902, a second row 904, and a third row 906. Similarly, the nodules 202 can be arranged in parallel columns. For example, the nodules 202 can be arranged in a first column 908, a second column 910, and a third column 912. In some embodiments, the nodules 202 that are adjacent to each other in each row can be equidistantly spaced from each other; similarly, the nodules 202 that are adjacent to each other in each column can be equidistantly spaced from each other. For example, a distance between centers of the nodules 202 in adjacent rows and columns can be about 15 mm. The nodules 202 in adjacent rows, for example, the first row 902 and the second row 904, can be joined by joins, such as the ribs 204; similarly, the nodules 202 in adjacent columns, for example, the first column 908 and the second column 910, can be joined by ribs 204. In some embodiments, the joins can be about 3 mm in width. An opening 414 can be disposed between every four nodules 202. For example, an opening 414 can be disposed between the nodule 202 in the first row 902 and the first column 908, the nodule 202 in the first row 902 and the second column 910, the nodule 202 in the second row 904 and the first column 908, and the nodule 202 in the second row 904 and the second column 910. Each nodule 202 may have the nodule ring 412. In some embodiments, the nodules ring 412 can be disposed about an equator of each nodule 202.

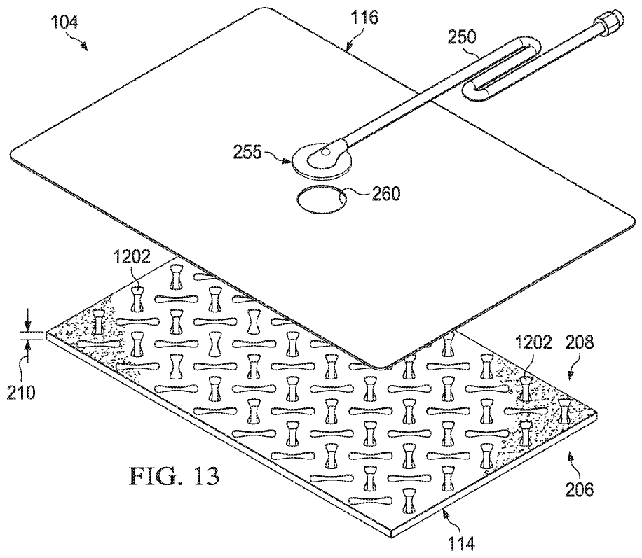

FIG. 13 is a perspective view illustrating additional details of another dressing 104 that may be associated with some embodiments of the therapy system 100 of FIG. 1. The dressing 104 can include the tissue interface 114, the cover 116, the fluid conductor 250, and the connector 255. The tissue interface 114 can have the first side 206, the second side 208 opposite the first side 206, and the thickness 210. In some embodiments, the tissue interface 114 may comprise a sheet of material having a plurality of openings 1202. The openings 1202 can extend through the tissue interface 114 from the first side 206 to the second side 208. In some embodiments, each opening 1202 may have an oblong shape. In some embodiments, each opening may have bulbous ends joined by a narrow portion.

In some embodiments, the thickness of the tissue interface 114 can be between about 10 mm and about 30 mm and, preferably, about 15 mm. The tissue interface 114 can be formed from an open-cell reticulated foam having a firmness factor/compression level/felting level of between about 5 and about 7. For example, the tissue interface 114 can be formed from an open-cell, reticulated foam that has been felted to have a density between about 5 times and about 7 times greater than the original density of the foam.

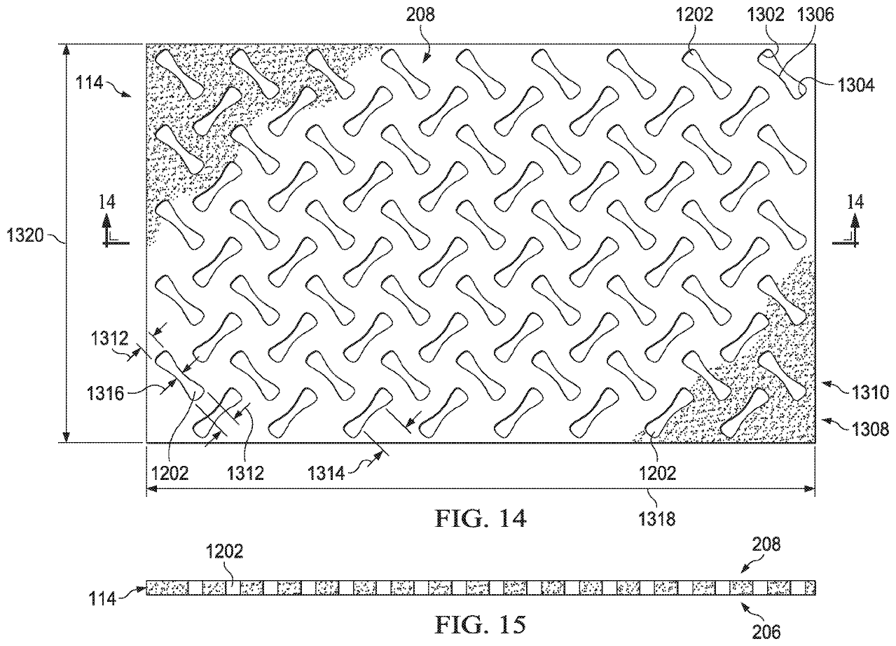

FIG. 14 is a plan view illustrating additional of the tissue interface 114 that may be associated with some embodiments of the dressing of FIG. 13. In some embodiments, the openings 1202 may have a length of between about 15 mm and about 20 mm. In some embodiments, each opening can have a first shoulder, such as a first end 1302, a second shoulder, such as a second end 1304, and a gauge section, such as a connecting portion 1306. The first end 1302 and the second end 1304 can be on opposite ends of the opening 1202. In some embodiments, each of the first end 1302 and the second end 1304 can have a first effective diameter 1312. In some embodiments, the first effective diameter 1312 can be about 5 mm. The connecting portion 1306 may have a length 1314 joining the first end 1302 to the second end 1304. In some embodiments, the connecting portion 1306 may have a width approximate equal to the average effective diameter of the first end 1302 and the second end 1304 proximate to the first end 1302 and the second end 1304. The width of the connecting portion can taper along the length 1314 from the first end 1302 and the second end 1304 to a minimum width 1316 at a point along the length 1314 that is substantially equidistant from the first end 1302 and the second end 1304. In some embodiments, the maximum width of the connecting portion 1306 can be about between about 5 mm. The minimum width 1316 of the connecting portion 1306 can be about 1 mm.

In some embodiments, the openings 1202 can be disposed in a herringbone-type pattern. In some embodiments, the pattern of the openings 1202 may extend from a first corner parallel to both a length 1318 and width 1320 of the tissue interface 114. In some embodiments, the openings 1202 may be oriented in rows parallel to the length 1318 of the tissue interface 114. For example, the tissue interface 114 may have at least a first row 1308 and a second row 1310. The length 1314 of each of the openings 1202 of the first row 1308 may be oriented relative to the length 1318 of the tissue interface 114 so that the length 1314 of the openings 1202 forms a 45-degree angle with the length 1318 of the tissue interface 114. The length 1314 of each of the openings 1202 of the second row 1310 may be oriented relative to the length 1314 of the openings 1202 of the first row 1308 so that the lengths 1314 of the openings 1202 of the second row 1310 are perpendicular to the lengths 1314 of the openings 1202 of the first row 1308. In other embodiments, the lengths 1314 of the openings 1202 of the first row 1308 may be oriented at other angles to the length 1318 of the tissue interface 114. Subsequent rows may be oriented similarly to the first row 1308 and the second row 1310.

FIG. 15 is a sectional view of the tissue interface of the tissue interface 114. Each of the openings 1202 may extend through the tissue interface 114 from the first surface 206 to the second surface 208. The openings 1202 provide a path for fluid communication across the tissue interface 114. The tissue interface 114 may be manufactured by providing a block of foam, for example, an open-cell reticulated block of foam. The block of foam may be felted to the appropriate felting level. Following felting, the openings 1202 may be formed in the block of foam to produce the tissue interface 114. For example, the openings 1202 may be formed by cutting the block of foam, vaporizing the foam, melting the foam, or otherwise removing portions of the foam in the pattern of the openings 1202.

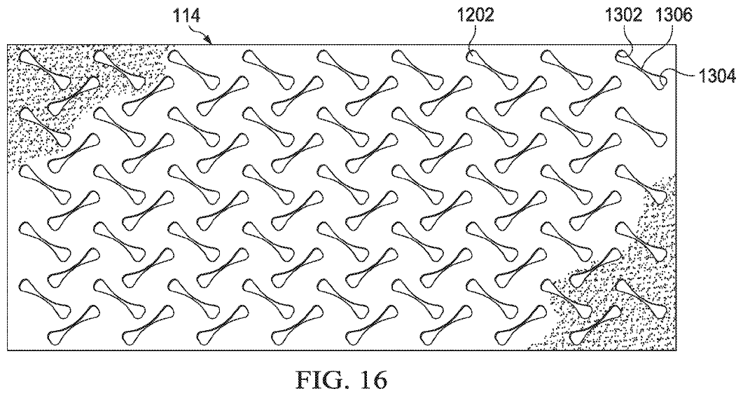

FIG. 16 is a plan view illustrating additional details of the tissue interface 114 of FIG. 14 that may be associated with some embodiments during application of negative pressure. In operation, the tissue interface 114 may be disposed at a tissue site. The tissue interface 114 can be covered with a cover 116 and fluidly coupled to the negative-pressure source 102. Fluid may be drawn from the sealed space formed by the cover 116. As fluid is drawn from the sealed space, a negative-pressure may be generated in the sealed space. In response to the decreasing pressure, the openings 1202 may collapse. In some embodiments, the connecting portion 1306 may completely collapse so that opposing sides of the opening 1202 at the connecting portion 1306 contact. Contact between the opposing sides of the opening 1202 may absorb or transmit load across the opening 1202 in response to an increase in force applied. As the connection portion 1306 absorbs the load, the first end 1302 and the second end 1304 may partially collapse or remain completely open. Preferably, the openings 1202 collapse so that the tissue interface collapse radially. As a result, fluid flow may continue across the tissue interface 114 from the first side 206 to the second side 208.

The systems, apparatuses, and methods described herein may provide significant advantages. For example, the tissue interface described herein can collapse without suffering ingrowth and still hold the tissue site open laterally. The tissue interface may also improve fluid delivery and removal. The tissue interface can have a long wear time without suffering in-growth while allowing for apposition under the application of negative pressure. The tissue interface can transport thick exudates and provide improved delivery of fluids to the tissue site if used with instillation therapy. The tissue interface may be stronger than other dressings and used with tunnel tissue sites. Furthermore the tissue interface may be easily conformed to tissue sites and sized by the end user.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A tissue interface for treating a tissue site, the tissue interface comprising:
a plurality of shapes; and
a plurality of ribs, each rib of the plurality of ribs having a first end coupled to a respective shape of the plurality of shapes and a second end coupled to at least one other rib of the plurality of ribs.

2.-8. (canceled)

9. The tissue interface of claim 7, wherein each shape of the plurality of shapes comprises a felted open-cell foam and a density of the felted foam is between 5 times and 7 times the density of the un-felted foam.

10. The tissue interface of claim 7, wherein each shape of the plurality of shapes comprises a felted open-cell foam and each shape of the plurality of shapes has a firmness factor between about 5 and about 7.

11. The tissue interface of claim 7, wherein each shape of the plurality of shapes comprises a felted open-cell foam and each shape of the plurality of shapes has a density of rubber.

12. (canceled)

13. The tissue interface of claim 1, wherein the tissue interface is configured to collapse laterally in response to an application of negative-pressure to the tissue interface.

14. The tissue interface of claim 1, wherein the tissue interface has a surface area and the surface area decreases by about 30% in response to an application of negative-pressure to the tissue interface.

15. The tissue interface of claim 1, wherein the first end of each rib of the plurality of ribs is tangentially coupled to a surface of the respective shape of the plurality of shapes.

16. The tissue interface of claim 1, wherein each rib of the plurality of ribs may have a width between about 1 mm and about 4 mm, a thickness up to about 3 mm, and a length between about 1 mm and about 10 mm.

17. The tissue interface of claim 1, wherein each rib of the plurality of ribs comprises a felted foam.

18. The tissue interface of claim 1, further comprising a plurality of holes formed between the plurality of ribs, each hole of the plurality of holes bounded by at least one respective rib of the plurality of ribs.

19. (canceled)

20. (canceled)

21. The tissue interface of claim 1, wherein the plurality of shapes and the plurality of ribs are formed from a closed-cell foam.

22. The tissue interface of claim 21, wherein the plurality of shapes are perforated.

23. (canceled)

24. (canceled)

25. A tissue interface for treating a tissue site, the tissue interface comprising:
a sheet of felted open-cell foam; and
a plurality of holes formed in the sheet, each hole extending through the sheet, each hole having a first end and a second end joined by a gauge section, the first end and the second end forming shoulders that are wider than the gauge section.

26. A system for treating a tissue site with negative-pressure, the system comprising:
a manifold configured to be disposed adjacent to the tissue site, the manifold having:
a plurality of nodules, and
a plurality of webs, each web of the plurality of webs having a first end coupled to a respective nodule of the plurality of nodules and a second end coupled to at least one other web of the plurality of webs;
a sealing member configured to be disposed over the manifold and to seal to tissue surrounding the tissue site; and
a negative-pressure source configured to be fluidly coupled to the manifold and operable to draw fluid through the manifold.

27. The system of claim 26, wherein each nodule of the plurality of nodules is spherical.

28. The system of claim 26, wherein each nodule of the plurality of nodules is conical.

29. The system of claim 26, wherein each nodule of the plurality of nodules is polyhedral.

30.-36. (canceled)

37. The system of claim 26, wherein the each nodule of the plurality of nodules has an average effective diameter between about 5 mm and about 20 mm.

38.-43. (canceled)

44. The system of claim 26, wherein the plurality of nodules are formed from a film.

45. The system of claim 26, wherein the plurality of webs are formed from a polymeric film.

46.-64. (canceled)
